(19) United States
(12) Patent Application Publication
(10) Pub. No.: US 2021/0299116 A1
(43) Pub. Date: Sep. 30, 2021

(54) METHODS FOR REDUCING ABNORMAL SCAR FORMATION

(71) Applicant: CORNELL UNIVERSITY, Ithaca, NY (US)

(72) Inventors: Randi B. SILVER, Ithaca, NY (US); Alexandria Rose SAVAGE, Ithaca (US)

(21) Appl. No.: 17/265,387

(22) PCT Filed: Aug. 2, 2019
(86) PCT No.: PCT/US2019/044785

§ 371 Date: Feb. 2, 2021

Related U.S. Application Data
(60) Provisional application No. 62/714,114, filed on Aug. 3, 2018.
(60) Provisional application No. 62/822,193, filed on Mar. 22, 2019.

(51) Int. Cl. A61K 31/472; A61K 31/4436; A61K 9/00; A61P 17/02

(52) U.S. Cl. CPC A61K 31/472; A61P 17/02; A61K 9/0014; A61K 31/4436

(57) ABSTRACT

This disclosure is directed to methods of reducing scar collagen abundance, scar width, and scar tissue contracture comprising administering to a subject in need of such treatment an effective amount of a mast cell stabilizer, or an effective amount of a HIF prolyl hydroxylase domain inhibitor, or an effective amount of a combination of a mast cell stabilizer and a HIF prolyl hydroxylase domain inhibitor.

### CROSS REFERENCE TO RELATED APPLICATION

This application claims the benefit of priority from U.S. Provisional Application No. 62/714,114, filed Aug. 3, 2018, and U.S. Provisional Application No. 62/822,193, filed Mar. 22, 2019, the entire contents of which are incorporated herein by reference.

### BACKGROUND

It is estimated that each year about 100 million people in the developed world develop scars after surgical operations. Of these, studies suggest that 40% to 70% result in hypertrophic scarring and a further 6% to 16%, primarily in African populations, develop keloids (an area of irregular fibrous tissue formed at the site of a scar or injury). These scars have a fibrotic phenotype marked by excessive collagen deposition. This abnormal scarring can lead to pruritus (severe itching of the skin), pain and contractures, and be cosmetically undesirable, all resulting in a negative quality of life. The biological processes involved in wound healing are very complex undergoing four distinct phases involving multiple cell types. Currently used therapies have limited effectiveness and/or are associated with significant side effects.

Mast cells reside in the dermis layer of skin and contribute to the inflammatory response to skin injury. When the skin is injured, mast cells become activated, degranulate, and release a large number of mediators with a wide range of biological activities. As a result, multiple roles for mast cells in wound healing have been described including recruitment of circulating inflammatory cells, promoting re-epithelialization and stimulating angiogenesis. Despite the knowledge that mast cells are involved in many aspects of wound healing, their function in vivo is not clearly defined as in studies showing that mast cells are required for wound healing (Weller K. et al., FASEB Journal 20: 2366-2368, 2006) while others show they are not necessary for wound healing (Nauta A C., PloS One, 8: e59167, 2013). Murine wound healing studies using systemic disodium cromoglycate have produced mixed and conflicting results (Dabrowski R. et al., Acta Physiologica Polonica, 41: 195-198, 1990; Chen L. et al., PloS One, 9: e85226, 2014).

Fibroblasts, which synthesize collagen, also produce smooth muscle actin (SMA), a protein responsible for the transformation of fibroblasts to myofibroblasts, which can occur during wound healing, and scar contracture. SMA is induced in fibroblasts by the cytokine transforming growth factor beta 1 (TGF-β). SMA promotes fibroblast contraction leading to painful scar contracture.

### SUMMARY OF THE DISCLOSURE

An aspect of this disclosure is directed to a method for reducing scar collagen abundance, scar width, and scar tissue contracture comprising administering to a subject in need of such treatment an effective amount of a mast cell stabilizer.

In some embodiments, the effective amount of the mast cell stabilizer is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of mast cell stabilizer.

In some embodiments, the mast cell stabilizer is selected from the group consisting of cromoglicic acid, ketotifen, olopatadine, rupatadine, mepolizumab, omalizumab, pemirolast, quercetin, nedocromil, azelastine, tranilast, palmitoylethanolamide, and vitamin D.

In some embodiments, the mast cell stabilizer is ketotifen.

In some embodiments, the mast cell stabilizer is administered locally to a wound.

Another aspect of this disclosure is directed to a method for reducing scar collagen abundance, scar width, and scar tissue contracture comprising administering to a subject in need of such treatment an effective amount of a HIF prolyl hydroxylase domain inhibitor.

In some embodiments, the effective amount of the HIF prolyl hydroxylase domain inhibitor is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of HIF prolyl hydroxylase domain inhibitor.

In some embodiments, the HIF prolyl hydroxylase inhibitor is selected from the group consisting of Roxadustat (RXD) (FG-4592), Vadadustat (AKB-6548), Daprodustat (GSK-1278863), and Molidustat (BAY 85-3934).

In some embodiments, the HIF prolyl hydroxylase inhibitor is RXD. In some embodiments, RXD is administered systemically.

Yet another aspect of this disclosure is directed to a method for reducing scar collagen abundance, scar width, and scar tissue contracture comprising administering to a subject in need of such treatment a combination therapy comprising an effective amount of a mast cell stabilizer and an effective amount of a HIF prolyl hydroxylase domain inhibitor.

In some embodiments, the mast cell stabilizer and the HIF prolyl hydroxylase domain inhibitor are administered as one composition. In some embodiments, the mast cell stabilizer and the HIF prolyl hydroxylase domain inhibitor are administered separately. In some embodiments, the mast cell stabilizer and the HIF prolyl hydroxylase domain inhibitor are administered consecutively. In some embodiments, the mast cell stabilizer and the HIF prolyl hydroxylase domain inhibitor are administered simultaneously.

In some embodiments, the effective amount of the mast cell stabilizer is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of mast cell stabilizer.

In some embodiments, the effective amount of the HIF prolyl hydroxylase domain inhibitor is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of HIF prolyl hydroxylase domain inhibitor.

In some embodiments, the mast cell stabilizer is selected from the group consisting of cromoglicic acid, ketotifen, olopatadine, rupatadine, mepolizumab, omalizumab, pemirolast, quercetin, nedocromil, azelastine, tranilast, palmitoylethanolamide, and vitamin D.

In some embodiments, the mast cell stabilizer is ketotifen.

In some embodiments, the mast cell stabilizer is administered locally to an incision site.

In some embodiments, the HIF prolyl hydroxylase inhibitor is selected from the group consisting of Roxadustat (RXD) (FG-4592), Vadadustat (AKB-6548), Daprodustat (GSK-1278863), and Molidustat (BAY 85-3934).

In some embodiments, the HIF prolyl hydroxylase inhibitor is RXD.

In some embodiments, the HIF prolyl hydroxylase inhibitor is administered systemically.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. Mast cells are abundant in hypertrophic scars. Representative fixed human tissue specimens of (A) hypertrophic scar and (B) normal skin stained for mast cells using HRP-avidin.

FIG. 2. Mast cell deficiency lessens the inflammatory response. Analysis of immune cells in fixed H&E stained skin (CTR) and scar (WD) 7 days post-incision in mast-cell-deficient (MCD) and congenic control wild type (WT) mice. *=P<0.05 between CTR and WD, n=5 mice/group.

FIGS. 3A-3E. Mast cell deficiency lessens scar width and collagen content. Analysis of collagen (blue stain) in Gomori trichrome stained fixed normal skin (CTR) and scar (WD) 14 days post-incision in mast-cell-deficient (MCD) and congenic control wild type (WT) mice. (A) Wild type (WT) mouse, normal skin (CTR); (B) Wild type (WT) mouse, scar (WD); (C) Mast cell deficient (MCD) mouse, normal skin (CTR); (D) Mast cell deficient (MCD) mouse, scar (WD). (E) Quantification of collagen thickness of (A) (D). *=P<0.05 between CTR and WD, n=5 mice/group. NS=not significant.

FIGS. 4A-4B. (A)Tensile Strength of the scar is not affected by mast cell deficiency. Tensitometry measurement of tensile strength (peak tension measured in Newtons (N)) of scar (WD) 14 days post-incision in mast-cell-deficient (MCD) and congenic control wild type (WT) mice. N=5 mice/group. There is no significant difference between the 2 groups. (B) Mast cell deficiency improves the appearance of the scar. Representative photographs of dorsal scar 14 days post-incision in mast-cell-deficient (MCD) and congenic control wildtype (WT) mice.

FIG. 5. Treatment of wounds with a mast cell stabilizer (ketotifen) lessens the inflammatory response to the incisional wound. Analysis of immune cells in fixed H&E stained (non-damaged) skin (Skin) and scar (Wound) 7 days post-incision in C57bl/6 mice. **P<0.01 between Skin and Wound, and **P<0.01 between Wound and Wound+Drug. There was no difference in immune cells between Skin and Wound+Drug. n=5 mice/group.

FIGS. 6A-6B. Treatment with mast cell stabilizer decreases scar width and collagen content. (A) Histological sections of scar 14 days after the incision that was sutured with silk impregnated with ketotifen (right) or untreated silk (left). C: infiltrating inflammatory cells; E: Epidermis; F: Hair follicles; H: Hypodermis; M: Muscle. (B) Analysis of scar width in Gomori trichrome stained fixed wound and wound+drug (ketotifen) 14 days post-incision in C57bl/6 mice. *=P<0.05 between wound and wound+drug, n=5 mice/group.

FIG. 7. Mast cell stabilizer decreases mRNA expression of newly synthesized collagen in wound scar. Q-PCR analysis of collagen III mRNA in Skin, Wound, and Wound+Drug. ***P<0.001 between skin and wound and skin and wound+drug, respectively.

FIGS. 8A-8B. (A) Visualization of New Collagen with Birefringence Microscopy: New Collagen III (Green) and Old Collagen I (Red). Representative micrographs of skin, wound and wound+drug viewed with birefringence microscopy for the visualization of old (collagen 1, red) and new (collagen III, green) collagen. (B) Treatment with mast cell Stabilizer ketotifen decreases newly synthesized collagen (III) in wound scar. Graph showing the % change in newly synthesized collagen III as determined by birefringence microscopy in wound and wound+drug. *=P<0.05 between wound and wound+drug. N=4 mice/group.

FIG. 9. Tensile strength of the scar is not affected by mast cell stabilizer. Tensitometry measurement of tensile strength. Peak tension measured in Newtons (N)) of scar and scar+drug , 14 days post-incision. N=5 mice/group. There is no significant difference between the two groups.

FIGS. 10A-10C. (A) Roxadustat (RXD), an HIF prolyl hydroxylase domain inhibitor, significantly reduces smooth muscle actin (SMA) abundance in fibroblasts as determined by Western blot. ****=P<0.0001, n=4. (B) Roxadustat inhibits SMA expression in fibroblasts. (C) Representative 20× immunohistochemistry images of fibroblasts stained for vimentin (red), a fibroblast intermediate filament, and smooth muscle actin (SMA) (green). Nuclei are stained with DAPI (blue). The SMA staining observed with TGF-β (green) is inhibited with RXD.

FIG. 11A-11B. Roxadustat treatment reduces fibroblast contraction. (A) Roxadustat significantly reduces TGFP-induced fibroblast contracture. ***P<0.001 between control and TGF-β+RXD. Addition of RXD or TGFβ led to significant changes (*P<0.05) from control. N=3 separate experiments. (B) Representative image of gel contraction assay.

FIG. 12. A schematic describing the use of ketotifen and/or Roxadustat to inhibit hypertrophic scarring.

FIG. 13. Model of mast-cell-dependent mechanism in hypertrophic scarring.

### DETAILED DESCRIPTION

### Descriptions

As used herein, the term “scar” refers to an area of fibrous tissue that replaces normal skin after an injury. In some embodiments, the scar is a hypertrophic scar. The phrase “hypertrophic scar” refers to a scar with collagen overproduction that causes the scar to be raised above the surrounding skin. In some embodiments, the scar is a keloid scar. The phrase “keloid scar” refers to more serious form of excessive scarring that can grow indefinitely into large, tumorous (although benign) neoplasms.

As used herein, the phrase “scar tissue contracture” refers to a tightening of the skin on or around the scar. Scar tissue contracture occurs as a result of contractile wound healing processes that occur in a scar that has already been re-epithelialized and adequately healed. In some embodiments, scar contracture occurs after a second or third degree burn.

### General Disclosure

### Methods for Reducing Scar Collagen Abundance, Scar Width, and Scar Tissue Contracture

One aspect of the present disclosure is directed to a method for reducing scar collagen abundance, scar width, and scar tissue contracture by administering to a subject in need of such treatment an effective amount of a mast cell stabilizer. In some embodiments, administering to a subject an effective amount of a mast cell stabilizer prevents or reduces collagen abundance, width, and scar tissue contracture.

In some embodiments, the mast cell stabilizer is administered locally to an incision where scar is expected to form. In some embodiments, the mast cell stabilizer is administered as impregnated in wound closures (sutures or stitches), in a cream, in a gel or in a bandage. In some embodiments, the mast cell stabilizer is administered systemically.

In some embodiments, an effective amount of a mast cell stabilizer is about 0.2 mg/kg to 100 mg/kg. In other embodiments, the effective amount of a mast cell stabilizer is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of mast cell stabilizer. As used in this disclosure, the term “about” refers to a variation within approximately ±10% from a given value.

Another aspect of the present disclosure is directed to a method for reducing scar collagen abundance, scar width, and scar tissue contracture by administering to a subject in need of such treatment an effective amount of a HIF prolyl hydroxylase domain inhibitor. In some embodiments, administering to a subject an effective amount of a HIF prolyl hydroxylase domain inhibitor prevents or reduces collagen abundance, width, and scar tissue contracture.

In some embodiments, the HIF prolyl hydroxylase domain inhibitor is administered locally to an incision where scar is expected to form. In some embodiments, the HIF prolyl hydroxylase domain inhibitor is administered as impregnated in wound closures (sutures or stitches), in a cream, in a gel or in a bandage. In some embodiments, the HIF prolyl hydroxylase domain inhibitor is administered systemically.

In some embodiments, an effective amount of a HIF prolyl hydroxylase domain inhibitor is about 0.2 mg/kg to 100 mg/kg. In other embodiments, the effective amount of a HIF prolyl hydroxylase domain inhibitor is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of HIF prolyl hydroxylase domain inhibitor.

A third aspect of the present disclosure is directed to a method for reducing scar collagen abundance, scar width, and scar tissue contracture by administering to a subject in need of such treatment a combination therapy comprising an effective amount of a mast cell stabilizer and an effective amount of a HIF prolyl hydroxylase domain inhibitor. In some embodiments, administering to a subject a combination therapy comprising an effective amount of a mast cell stabilizer and an effective amount of a HIF prolyl hydroxylase domain inhibitor prevents or reduces collagen abundance, width, and scar tissue contracture.

In some embodiments, the mast cell stabilizer and the HIF prolyl hydroxylase domain inhibitor are administered in one composition. In some embodiments, the mast cell stabilizer and the HIF prolyl hydroxylase domain inhibitor are administered consecutively. In some embodiments, the mast cell stabilizer and the HIF prolyl hydroxylase domain inhibitor are administered separately. In some embodiments, the mast cell stabilizer and the HIF prolyl hydroxylase domain inhibitor are administered simultaneously.

In some embodiments, the mast cell stabilizer and the HIF prolyl hydroxylase domain inhibitor are administered in one composition. In one embodiment, a composition comprising a mast cell stabilizer and a HIF prolyl hydroxylase domain inhibitor is not administered to the subject continuously; rather it is administered intermittently. In a specific embodiment, intermittent administration is performed once every other day, every three days, every four days, every five days, or once a week. In another specific embodiment, intermittent administration is performed once every hour, every two hours, every three hours, every six hours, every ten hours, or every twelve hours.

### Mast Cell Stabilizers

In some embodiments, the phrase “mast cell stabilizer” refers to an agent which prevents mast cells from breaking open and releasing chemicals (e.g., histamine, proteoglycans, serotonin, and serine proteases) that help cause inflammation (aka. “degranulation”). In some embodiments, the mast cell stabilizer is a small molecule. As used in this disclosure, the phrase “small molecule compound” refers to small organic chemical compound, generally having a molecular weight of less than 2000 daltons, 1500 daltons, 1000 daltons, 800 daltons, or 600 daltons.

In some embodiments, the mast cell stabilizer comprises compounds including, but not limited to

Cromoglycic acid (cromolyn) with the chemical formula C₂₃H₁₆O₁₁ and the chemical structure

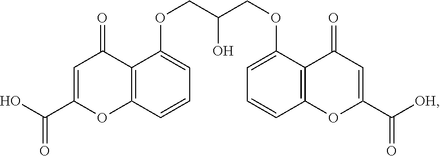 [SMILES: O=C(O)c1cc(=O)c2c(OCC(O)COc3cccc4oc(C(=O)O)cc(=O)c34)cccc2o1]

ketotifen with the chemical formula C₁₉H₁₉NOS and the chemical structure

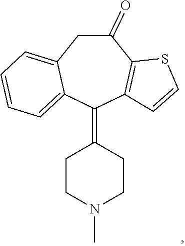 [SMILES: CN1CCC(=C2c3ccccc3CC(=O)c3sccc32)CC1]

pemirolast with the chemical formula C₁₀H₈N₆O and the chemical structure

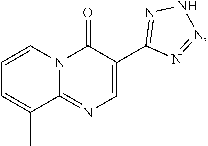 [SMILES: Cc1cccn2c(=O)c(-c3nn[nH]n3)cnc12]

and nedocromil with the chemical formula C₁₉H₁₇NO₇ and the chemical structure

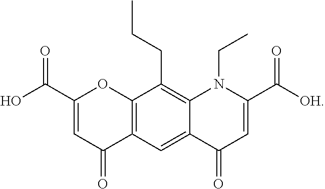 [SMILES: CCCc1c2oc(C(=O)O)cc(=O)c2cc2c(=O)cc(C(=O)O)n(CC)c12]

In some embodiments, the mast stabilizer is selected from the group consisting of Cromoglicic acid, Ketotifen, Olopatadine, Rupatadine, Mepolizumab, Omalizumab, Pemirolast, Quercetin, Nedocromil, Azelastine, Tranilast, Palmitoylethanolamide, and Vitamin D.

In some embodiments, the mast cell stabilizer is ketotifen. In some embodiments, ketotifen is administered locally to an incision where scar is expected to form. In some embodiments, ketotifen is administered systemically.

### HIF Prolyl Hydroxylase Domain Inhibitors

In some embodiments, the phrase “HIF prolyl hydroxylase domain inhibitor” refers to an agent that inhibits the activity of HIF hydroxylase or other enzymes in the 2-OG (2-oxoglutarate)-dependent dioxygenase enzyme superfamily having structural similarity and a common reaction mechanism with HIF hydroxylase. An agent that inhibits HIF prolyl hydroxylase activity is any agent that reduces or otherwise modulates the activity of a HIF prolyl hydroxylase or related enzyme, such as C—P4H. In some embodiments, the HIF prolyl hydroxylase domain inhibitor is a small molecule.

In some embodiments, the HIF prolyl hydroxylase domain inhibitor is selected from the group consisting of Roxadustat (RXD) (FG-4592), Vadadustat (AKB-6548), Daprodustat (GSK-1278863), and Molidustat (BAY 85-3934).

In some embodiments, the general structure of HIF prolyl hydroxylase domain inhibitors is

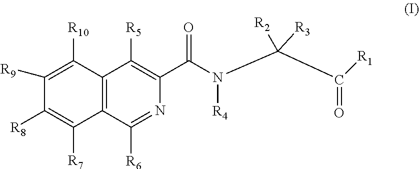

wherein:
R1 is selected from the group consisting of hydroxyl, alkoxy, substituted alkoxy, aryloxy, and substituted aryloxy;
R2 and R3 are independently selected from the group consisting of hydrogen, alkyl, substituted alkyl, cycloalkyl, substituted cycloalkyl, aryl, substituted aryl, heterocycloalkyl, substituted heterocycloalkyl, heteroaryl, and substituted heteroaryl; or R2 and R3 together with the carbon atom to which they are attached form a cycloalkyl, substituted cycloalkyl, hererocycloalkyl, or substituted hererocycloalkyl;
R4 is selected from the group consisting of hydrogen, alkyl, and substituted alkyl;
R5 is selected from the group consisting of hydroxyl, alkoxy, and substituted alkoxy;
R6 is selected from the group consisting of hydrogen, alkyl, substituted alkyl, cycloalkyl, substituted cycloalkyl, cyano, halo, hydroxyl, alkoxy, substituted alkoxy, amino, substituted amino, arylozy, substituted arylozy, aminoacyl, substituted aminoacyl, cycloalkoxy, substituted cycloalkoxy, aryl, substituted aryl, aryloxy, substituted aryloxy, heterocycloalkyl, substituted heterocycloalkyl, heteroaryloxy, substituted heteroaryloxy, heteroaryl, and substituted heteroaryl; and
R7, R8, R9 and R10 are independently selected from the group consisting of hydrogen, halo, alkyl, substituted alkyl, cycloalkyl, substituted cycloalkyl, aryl, substituted aryl, heteroaryl, substituted heteroaryl, alkoxy, substituted alkoxy, aryloxy, and substituted aryloxy.

In some embodiments, R6 is alkyl. In another embodiment, the alkyl at R6 is methyl.

In some embodiments, R5 is hydroxyl.

In some embodiments, R8 is aryloxy.

In some embodiments, wherein R6 is methyl and R5 is hydroxyl.

As used herein, the term “alkyl” refers to a straight or branched, saturated hydrocarbon group having 1 to 10 carbon atoms, more particularly from 1 to 5 carbon atoms, and even more particularly 1 to 3 carbon atoms. Representative alkyl groups include methyl, ethyl, n-propyl, iso-propyl, n-butyl, t-butyl, n-pentyl, and the like.

The term “substituted alkyl” refers to an alkyl group of from 1 to 10 carbon atoms, more particularly 1 to 5 carbon atoms, and having from 1 to 5 substituents, preferably 1 to 3 substituents, each of which substituents is independently selected from the group consisting of alkoxy, substituted alkoxy, acyl, acylamino, acyloxy, amino, substituted amino, aminoacyl, aminocarbonylamino, aminothiocarbonylamino, aminocarbonyloxy, aryl, substituted aryl, aryloxy, substituted aryloxy, aryloxyaryl, substituted aryloxyaryl, cyano, halogen, hydroxyl, nitro, oxo, thioxo, carboxyl, carboxyl esters, cycloalkyl, substituted cycloalkyl, thio, alkylthio, substituted alkylthio, arylthio, substituted arylthio, cycloalkylthio, substituted cycloalkylthio, heteroarylthio, substituted heteroarylthio, heterocyclicthio, substituted heterocyclicthio, sulfonyl, substituted sulfonyl, heteroaryl, substituted heteroaryl, heterocyclic, substituted heterocyclic, cycloalkoxy, substituted cycloalkoxy, heteroaryloxy, substituted heteroaryloxy, heterocyclyloxy, substituted heterocyclyloxy, oxycarbonylamino, oxythiocarbonylamino, —OS(O)2-alkyl, —OS(O)2-substituted alkyl, —OS(O)2-aryl, —OS(O)2-substituted aryl, —OS(O)2-heteroaryl, —OS(O)2-substituted heteroaryl, —OS(O)2-heterocyclic, —OS(O)2-substituted heterocyclic, and —OSO2NR11R11, —NR11S(O)2NR11-alkyl, —NR11S(O)2NR11-substituted alkyl, —NR11S(O)2NR11-aryl, —NR11S(O)2NR11-substituted aryl, —NR11S(O)2NR11-heteroaryl, —NR11S(O)2NR11-substituted heteroaryl, —NR11S(O)2NR11-heterocyclic, and —NR11S(O)2NR11-substituted heterocyclic, wherein each R11 is independently selected from hydrogen or alkyl. Representative substituted alkyl groups include trifluoromethyl, benzyl, pyrazol-1-ylmethyl and the like.

The term “alkoxy” refers to the group “alkyl-O—,” which includes, by way of example, methoxy, ethoxy, n-propoxy, iso-propoxy, n-butoxy, t-butoxy, sec-butoxy, n-pentoxy, and the like.

The term “substituted alkoxy” refers to the group “substituted alkyl-O—”.

The term “acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, provided that a nitrogen atom of the heterocyclic or substituted heterocyclic is not bound to the —C(O)— group, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminoacyl” or “amide”, or the prefix “carbamoyl,” “carboxamide,” “substituted carbamoyl” or “substituted carboxamide” refers to the group —C(O)NR12R12, wherein each R12 is independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or wherein each R12 is joined to form together with the nitrogen atom a heterocyclic or substituted heterocyclic, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “alkenyl” refers to a vinyl unsaturated monovalent hydrocarbyl group having from 2 to 6 carbon atoms, and preferably 2 to 4 carbon atoms, and having at least 1, and preferably from 1 to 2 sites of vinyl (>C═C<) unsaturation. Representative alkenyl groups include vinyl (ethen-1-yl), allyl, but-3-enyl and the like.

The term “substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxyl, carboxyl esters, cycloalkyl, substituted cycloalkyl, heteroaryl, substituted heteroaryl, heterocyclic, and substituted heterocyclic. This term includes both E (trans) and Z (cis) isomers as appropriate. It also includes mixtures of both E and Z components.

The term “alkynyl” refers to acetylenic unsaturated monovalent hydrocarbyl groups having from 2 to 6 carbon atoms, and preferably 2 to 3 carbon atoms, and having at least 1, and preferably from 1 to 2 sites of acetylenic (—C≡C—) unsaturation. Representative alkynyl groups include ethyn-1-yl, propyn-1-yl, propyn-2-yl, and the like.

The term “amino” refers to the group —NH2.

The term “substituted amino” refers to the group —NR13R13, wherein each R13 is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, substituted heterocyclic, sulfonyl, and substituted sulfonyl, provided that both R13 groups are not hydrogen; or the R13 groups can be joined together with the nitrogen atom to form a heterocyclic or substituted heterocyclic ring. Representative substituted amino groups include phenylamino, methylphenylamino, and the like. Representative substituted amino groups include (ethanic acid-2-yl)amino, and the like.

The term “acylamino” refers to the groups —NR14C(O)alkyl, —NR14C(O)substituted alkyl, —NR14C(O)cycloalkyl, —NR14C(O)substituted cycloalkyl, —NR14C(O)alkenyl, —NR14C(O)substituted alkenyl, —NR14C(O)alkynyl, —NR14C(O)substituted alkynyl, —NR14C(O)aryl, —NR14C(O)substituted aryl, —NR14C(O)heteroaryl, —NR14C(O)substituted heteroaryl, —NR14C(O)heterocyclic, and —NR14C(O)substituted heterocyclic, wherein R14 is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are defined herein.

The term “oxycarbonylamino” refers to the groups —NR15C(O)O-alkyl, —NR15C(O)O-substituted alkyl, —NR15C(O)O-alkenyl, —NR15C(O)O-substituted alkenyl, —NR15C(O)O-alkynyl, —NR15C(O)O-substituted alkynyl, —NR15C(O)O-cycloalkyl, —NR15C(O)O-substituted cycloalkyl, —NR15C(O)O-aryl, —NR15C(O)O-substituted aryl, —NR15C(O)O-heteroaryl, —NR15C(O)O-substituted heteroaryl, —NR15C(O)O-heterocyclic, and —NR15C(O)O-substituted heterocyclic, wherein R15 is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “oxythiocarbonylamino” refers to the groups —NR16C(S)O-alkyl, —NR16C(S)O-substituted alkyl, —NR16C(S)O-alkenyl, —NR16C(S)O-substituted alkenyl, —NR16C(S)O-alkynyl, —NR16C(S)O-substituted alkynyl, —NR16C(S)O-cycloalkyl, —NR16C(S)O-substituted cycloalkyl, —NR16C(S)O-aryl, —NR16C(S)O-substituted aryl, —NR16C(S)O-heteroaryl, —NR16C(S)O-substituted heteroaryl, —NR16C(S)O-heterocyclic, and —NR16C(S)O-substituted heterocyclic, wherein R16 is hydrogen or alkyl, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminocarbonyloxy,” or the prefix “carbamoyloxy” or “substituted carbamoyloxy,” refers to the groups —OC(O)NR17R17, wherein each R17 is independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic; or wherein each R17 is joined to form, together with the nitrogen atom, a heterocyclic or substituted heterocyclic, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

The term “aminocarbonylamino” refers to the group —NR18C(O)—NR18R18, wherein each R18 is independently selected from the group consisting of hydrogen and alkyl.

The term “aminothiocarbonylamino” refers to the group —NR19C(S)—NR19R19, wherein each R19 is independently selected from the group consisting of hydrogen and alkyl.

The term “aryl” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The term “substituted aryl” refers to aryl groups, as defined herein, which are substituted with from 1 to 4, particularly 1 to 3, substituents selected from the group consisting of hydroxyl, acyl, acylamino, acyloxy, alkyl, substituted alkyl, alkoxy, substituted alkoxy, alkenyl, substituted alkenyl, alkynyl, substituted alkynyl, amidino (—C(═NH)-amino or substituted amino), amino, substituted amino, aminoacyl, aminocarbonyloxy, aminocarbonylamino, aminothiocarbonylamino, aryl, substituted aryl, aryloxy, substituted aryloxy, cycloalkoxy, substituted cycloalkoxy, heteroaryloxy, substituted heteroaryloxy, heterocyclyloxy, substituted heterocyclyloxy, carboxyl, carboxyl esters, cyano, thio, alkylthio, substituted alkylthio, arylthio, substituted arylthio, heteroarylthio, substituted heteroarylthio, cycloalkylthio, substituted cycloalkylthio, heterocyclicthio, substituted heterocyclicthio, cycloalkyl, substituted cycloalkyl, guanidino (—NH—C(═NH)-amino or substituted amino), halo, nitro, heteroaryl, substituted heteroaryl, heterocyclic, substituted heterocyclic, oxycarbonylamino, oxythiocarbonylamino, sulfonyl, substituted sulfonyl, —OS(O)₂-alkyl, —OS(O)₂-substituted alkyl, —OS(O)₂-aryl, —OS(O)₂-substituted aryl, —OS(O)2-heteroaryl, —OS(O)₂-substituted heteroaryl, —OS(O)₂-heterocyclic, —OS(O)₂-substituted heterocyclic, and —OSO₂—NR20R20, —NR20S(O)₂—NR20-alkyl, —NR20S(O)₂—NR20-substituted alkyl, —NR20S(O)₂—NR20-aryl, —NR20S(O)₂—NR20-substituted aryl, —NR20S(O)₂—NR20-heteroaryl, —NR20S(O)₂—NR20-substituted heteroaryl, —NR20S(O)₂—NR20-heterocyclic, —NR20S(O)₂—NR20-substituted heterocyclic, wherein each R20 is independently selected from hydrogen or alkyl, and wherein each of the terms is as defined herein. Representative substituted aryl groups include 4-fluorophenyl, 3-methoxyphenyl, 4-t-butylphenyl, 4-trifluoromethylphenyl, 2-trifluoromethoxyphenyl, 3-trifluoromethoxyphenyl, 4-trifluoromethoxyphenyl, 2-chlorophenyl, 3-chlorophenyl, 4-chlorophenyl, 2-chloro-6-fluorophenyl, 2,4-dichlorophenyl, 4-methoxyphenyl, 3-cyanophenyl, 4-cyanophenyl, 4-phenoxyphenyl, 4-methanesulfonylphenyl, biphenyl-4-yl, and the like.

The term “aryloxy” refers to the group aryl-O— that includes, by way of example, phenoxy, naphthoxy and the like.

The term “substituted aryloxy” refers to substituted aryl-O— groups.

The term “aryloxyaryl” refers to the group -aryl-O-aryl.

The term “substituted aryloxyaryl” refers to aryloxyaryl groups substituted with from 1 to 3 substituents on either or both aryl rings as defined above for substituted aryl.

The term “carboxyl” refers to —COOH or salts thereof.

The term “carboxyl ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)β-cycloalkyl, —C(O)O-substituted cycloalkyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic.

The term “cyano” refers to the group —CN.

The term “cycloalkyl” refers to a saturated or an unsaturated but nonaromatic cyclic alkyl groups of from 3 to 10, 3 to 8 or 3 to 6 carbon atoms having single or multiple cyclic rings including, by way of example, adamantyl, cyclopropyl, cyclobutyl, cyclopentyl, cyclooctyl, cyclohexenyl, and the like.

The term “substituted cycloalkyl” refers to a cycloalkyl group, having from 1 to 5 substituents selected from the group consisting of oxo (═O), thioxo (═S), alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxy, nitro, carboxyl, carboxyl esters, cycloalkyl, substituted cycloalkyl, heteroaryl, substituted heteroaryl, heterocyclic, and substituted heterocyclic.

The term “cycloalkylene” and “substituted cycloalkylene” refer to divalent cycloalkyl and substituted cycloalkyl groups as defined above.

The term “cycloalkoxy” refers to —O-cycloalkyl groups.

The term “substituted cycloalkoxy” refers to —O-substituted cycloalkyl groups.

The term “halo” or “halogen” refers to fluoro, chloro, bromo, and iodo.

The term “hydroxy” or “hydroxyl” refers to the group —OH.

The term “heteroaryl” refers to an aromatic ring of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl) provided the point of attachment is through a ring containing the heteroatom and that ring is aromatic. The nitrogen and/or sulfur ring atoms can optionally be oxidized to provide for the N-oxide or the sulfoxide, and sulfone derivatives. Representative heteroaryl groups include pyridinyl, pyrimidinyl, pyrrolyl, pyrazolyl, indolyl, thiophenyl, thienyl, furyl, and the like.

The term “substituted heteroaryl” refers to heteroaryl groups that are substituted with from 1 to 3 substituents selected from the same group of substituents defined for substituted aryl. Representative substituted heteroaryl groups include 5-fluoro-pyridin-3-yl, 1-benzyl-1H-[1,2,3]triazol-4-yl, 5-bromo-furan-2-yl, trifluoromethyl-2H-pyrazol-3-yl, and the like.

The term “heteroaryloxy” refers to the group —O-heteroaryl, and “substituted heteroaryloxy” refers to the group —O-substituted heteroaryl.

The term “heterocyclyl” or “heterocyclic” refers to a saturated or unsaturated (but not aromatic) group having a single ring or multiple condensed rings, from 1 to 10 carbon atoms, and from 1 to 4 hetero atoms selected from the group consisting of nitrogen, sulfur or oxygen within the ring, wherein in fused ring systems, one or more of the rings can be aryl or heteroaryl provided that the point of attachment is at the heterocycle. The nitrogen and/or sulfur ring atoms can optionally be oxidized to provide for the N-oxide or the sulfoxide, and sulfone derivatives.

The term “substituted heterocyclyl” or “substituted heterocyclic” refers to heterocycle groups that are substituted with from 1 to 3 of the same substituents as defined for substituted cycloalkyl.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “nitro” refers to the group —NO₂.

The term “oxo” refers to the atom (═O) or to the atom (—O—).

The term “sulfonyl” refers to the group —S(O)₂H.

The term “substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein. Representative sulfonyl groups include methyl-SO₂—, phenyl-SO₂—, 4-methylphenyl-SO₂—, and the like.

The term “heterocyclyloxy” refers to the group —O-heterocyclic, and “substituted heterocyclyloxy” refers to the group —O-substituted heterocyclic.

The term “thio” refers to the group —SH.

The term “alkylsulfanyl,” “alkylthio,” or “thioether” refers to the groups —S-alkyl, wherein alkyl is as defined above.

The term “substituted alkylthio,” “substituted alkyl sulfanyl, ” or “substituted alkylthio” refers to the group —S-substituted alkyl, wherein substituted alkyl is as defined above.

The term “cycloalkylthio” or “cycloalkylsulfanyl” refers to the groups —S-cycloalkyl wherein cycloalkyl is as defined above.

The term “substituted cycloalkylthio” refers to the group —S-substituted cycloalkyl wherein substituted cycloalkyl is as defined above.

The term “arylthio” or “arylsulfanyl” refers to the group —S-aryl, and “substituted arylthio” refers to the group —S-substituted aryl, wherein aryl and substituted aryl are as defined above.

The term “heteroarylthio” or “heteroarylsulfanyl” refers to the group —S-heteroaryl, and “substituted heteroarylthio” refers to the group —S-substituted heteroaryl, wherein heteroaryl and substituted heteroaryl are as defined above.

The term “heterocyclicthio” or “heterocyclicsulfanyl” refers to the group —S-heterocyclic, and “substituted heterocyclicthio” refers to the group —S-substituted heterocyclic wherein heterocyclic, and substituted heterocyclic are as defined above.

The term “ester” refers to the group —C(O)OR21, wherein R21 is alkyl, substituted alkyl, aryl, or substituted aryl.

In one embodiment, in Formula (I), R1 and R5 are hydroxyl; R2, R3, R4, R7, R9, and R10 are hydrogen; R6 is methyl; and R8 is phenox, and the compound has a structure shown below in formula (II). This molecule is also known as FG-4592 (aka. Roxadustat), which is an isoquinolone having the chemical name, N-[(4-hydroxy-1-methyl-7-phenoxyisoquinolin-3-yl)carbonyl]glycine)].

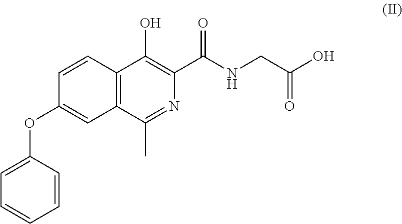 [SMILES: Cc1nc(C(=O)NCC(=O)O)c(O)c2ccc(Oc3ccccc3)cc12]

FG-4592 (Roxadustat) is in phase 3 clinical trials for the treatment of anemia in chronic kidney disease with no untoward effects reported.

In a specific embodiment, an effective amount of FG-4592 (Roxadustat) is an amount between 0.2 mg/kg and 20 mg/kg. In another embodiment an effective dosage of FG-4592 (Roxadustat) is 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 18 mg/kg, or 20 mg/kg.

In some embodiments, a FG-4592 (Roxadustat) analogue is used. FG-4592 analogues are described in U.S. Pat. Nos. 9,701,647; 9,439,888; 7,863,292; and U.S. patent application Ser. Nos. 13/186,351 and 11/549,571, which are all incorporated by reference in their entirety.

In some embodiments, the HIF prolyl hydroxylase domain inhibitor is RXD (roxadustat). In some embodiments, RXD is administered locally to an incision where scar is expected to form. In some embodiments, RXD is administered systemically.

### Pharmaceutical Compositions Comprising Mast Cell Stabilizer or a HIF Prolyl Hydroxylase Domain Inhibitor

In some embodiments, a mast cell stabilizer or a HIF prolyl hydroxylase domain inhibitor can be combined with a pharmaceutically acceptable carrier prior to administration. For the purposes of this disclosure, “pharmaceutically acceptable carriers” means any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, and the like. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

A mast cell stabilizer or a HIF prolyl hydroxylase domain inhibitor can be admixed with a pharmaceutically acceptable carrier to make a pharmaceutical preparation in any conventional form including, inter alia, a solid form such as tablets, capsules (e.g. hard or soft gelatin capsules), pills, cachets, powders, granules, and the like; a liquid form such as solutions, suspensions; or in micronized powders, sprays, aerosols and the like.

The pharmaceutical compositions of the present disclosure can be used in liquid, solid, tablet, capsule, pill, ointment, cream, nebulized or other forms as explained below. In some embodiments, the composition of the present disclosure may be administered by different routes of administration such as oral, oronasal, parenteral or topical.

“Oral” or “peroral” administration refers to the introduction of a substance into a subject's body through or by way of the mouth and involves swallowing or transport through the oral mucosa (e.g., sublingual or buccal absorption) or both.

“Oronasal” administration refers to the introduction of a substance into a subject's body through or by way of the nose and the mouth, as would occur, for example, by placing one or more droplets in the nose. Oronasal administration involves transport processes associated with oral and intranasal administration.

“Parenteral administration” refers to the introduction of a substance into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

“Topical administration” means the direct contact of a substance with tissue, such as skin or membrane, particularly the oral or buccal mucosa.

The pharmaceutical preparations of the present disclosure can be made up in any conventional form including, inter alia,: (a) a solid form for oral administration such as tablets, capsules (e.g. hard or soft gelatin capsules), pills, sachets, powders, granules, and the like; (b) preparations for topical administrations such as solutions, suspensions, ointments, creams, gels, micronized powders, sprays, aerosols and the like. The pharmaceutical preparations may be sterilized and/or may contain adjuvants such as preservatives, stabilizers, wetting agents, emulsifiers, salts for varying the osmotic pressure and/or buffers.

For topical administration to the skin or mucous membrane the aforementioned composition is preferably prepared as ointments, tinctures, creams, gels, solution, lotions, sprays; aerosols and dry powder for inhalation, suspensions, shampoos, hair soaps, perfumes and the like. In fact, any conventional composition can be utilized in this invention. Among the preferred methods of applying the composition containing the agents of this invention is in the form of an ointment, gel, cream, lotion, spray; aerosol or dry powder for inhalation. The pharmaceutical preparation for topical administration to the skin can be prepared by mixing the aforementioned active ingredient with non-toxic, therapeutically inert, solid or liquid carriers customarily used in such preparation. These preparations generally contain 0.01 to 5.0 percent by weight, or 0.1 to 1.0 percent by weight, of the active ingredient, based on the total weight of the composition.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the aforementioned active agent. Among the conventional antioxidants which can be utilized in these preparations are included N-methyl-a-tocopherolamine, tocopherols, butylated hydroxyanisole, butylated hydroxytoluene, ethoxyquin and the like.

Cream-based pharmaceutical formulations containing the active agent, used in accordance with this invention, are composed of aqueous emulsions containing a fatty acid alcohol, semi-solid petroleum hydrocarbon, ethylene glycol and an emulsifying agent.

Ointment formulations containing the active agent in accordance with this invention comprise admixtures of a semi-solid petroleum hydrocarbon with a solvent dispersion of the active material. Cream compositions containing the active ingredient for use in this invention preferably comprise emulsions formed from a water phase of a humectant, a viscosity stabilizer and water, an oil phase of a fatty acid alcohol, a semi-solid petroleum hydrocarbon and an emulsifying agent and a phase containing the active agent dispersed in an aqueous stabilizer-buffer solution. Stabilizers may be added to the topical preparation. Any conventional stabilizer can be utilized in accordance with this invention. In the oil phase, fatty acid alcohol components function as a stabilizer. These fatty acid alcohol components function as a stabilizer. These fatty acid alcohol components are derived from the reduction of a long-chain saturated fatty acid containing at least-14 carbon atoms.

Also, conventional perfumes and lotions generally utilized in topical preparation for the hair can be utilized in accordance with this invention. Furthermore, if desired, conventional emulsifying agents can be utilized in the topical preparations of this invention.

In some embodiments, compositions comprising a mast cell stabilizer or a HIF prolyl hydroxylase can be administered by aerosol. For example, this can be accomplished by preparing an aqueous aerosol, liposomal preparation or solid particles containing a composition comprising a mast cell stabilizer or a HIF prolyl hydroxylase preparation. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1

### Materials and Methods

### Animal Models of Scarring

As there are no murine models for hypertrophic or keloid scarring, the inventors relied on a classic rodent incisional wound model consisting of a longitudinal para-vertebral incision 1-2 cm long through the entire thickness of the skin and cutaneous muscle. Wounds (WD) and size matched pieces of dorsal dermis (CTR) were excised and processed for light and polarized light microscopy: fixation, embedding, sectioning and stained with hematoxylin-eosin (inflammatory cells) and Gomori Trichrome (collagen staining), tensile strength measurements and qPCR.

In some experiments, a drug was delivered locally and directly to the wound. In some embodiments, the drug was impregnated in the wound closure material, silk suture. In some experiments, the drug was eluted into the wound during wound healing leading to closure and scar formation. Wound healing was monitored over 14 days.

### Contraction Assay

Evaluation of fibroblasts contractility was performed as previously described by Pincha et al (Bio-protocol 8(18): e3021, 2018). Briefly, HLF cells are resuspended in 1 mg/ml collagen and then pipetted into 24 well plates. Collagen is allowed to solidify for 30 minutes at room temperature. Media (500 μl) is then added to each of the wells and the gels are dissociated from the walls of the well. Gels are treated and allowed to incubate for 48 hours at 37° C. and 5% CO₂. After the 48 hour incubation, gels are stained with crystal violet stain in order to better visualize contraction. Images are analyzed using Image J software and the fold change compared to a no cell control is determined.

### Tissue Harvest

Wounds (WD) and size matched pieces of dorsal dermis (CTR) were excised and processed for light microscopy: fixation, embedding, sectioning and stained with hematoxylin-eosin (inflammatory cells) and Gomori Trichrome (collagen staining).

### Example 2

### Mast Cells are Involved in Scar Tissue Formation

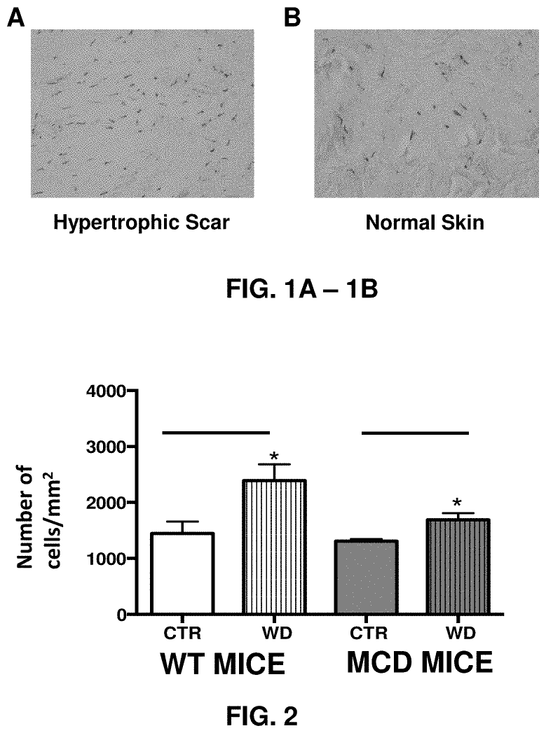

To determine the contribution of mast cells to incisional wound healing and scar formation experiments were performed with mast-cell-deficient WBB6F₁-W/W^{v} (MCD) mice and their congenic controls WBB6F₁-⁺/⁺W/W^{v} (WT) or the C57bl/6 mouse strain and a classic rodent incisional wound model consisting of a longitudinal para-vertebral incision 1-2 cm long through the entire thickness of the skin and cutaneous muscle. Mast cells were found abundantly in hypertrophic scars (FIGS. 1A and 1B).

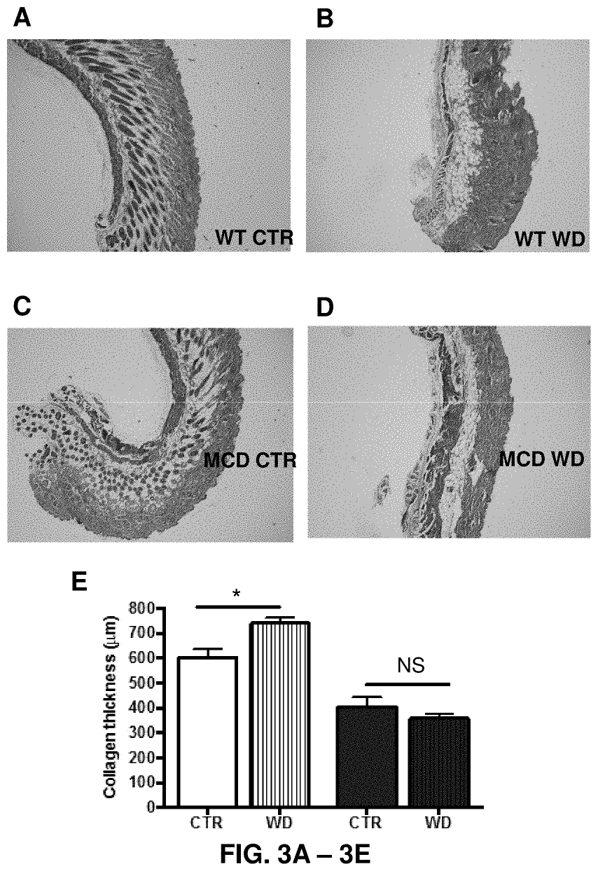

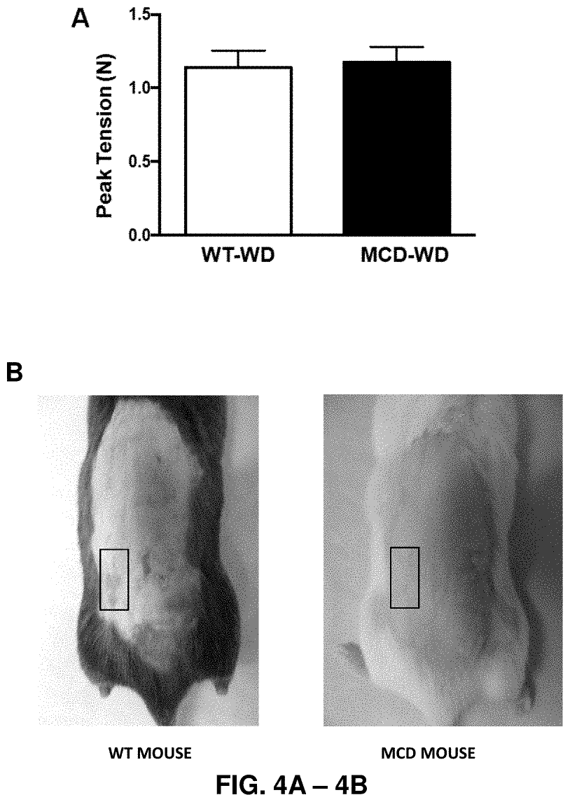

Mast-cell-deficiency lessened the inflammatory response to an incisional wound as shown at the 7-day time point compared to that measured in the wound of the congenic control wild-type mice (FIG. 2). Mast-cell-deficiency also decreased the scar width and collagen content of the scar as determined by Gomori stain (blue) in dermal layer-matched fixed sections of wound closure (scar) at the 14-day time point (FIGS. 3A-3E).

Peak tension measured in Newtons was determined on excised scars from MCD and WT wound (day 14) and found to be similar (FIG. 4A). Scars from the incisional wound in mast-cell-deficient mice had a better appearance compared to the wild-type scars (day 14) (FIG. 4B)

### Example 3

### Mast Cell Stabilization Prevents Scar Tissue Formation

Using wild type C57bl/6 mice, wound healing experiments were next performed where the incision was sutured with silk imbedded with ketotifen, a mast cell stabilizer. This way the drug could be delivered continuously and locally during wound healing.

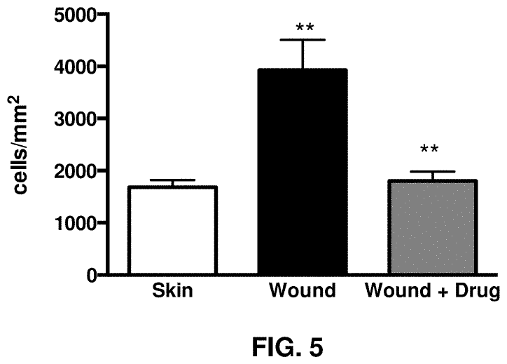

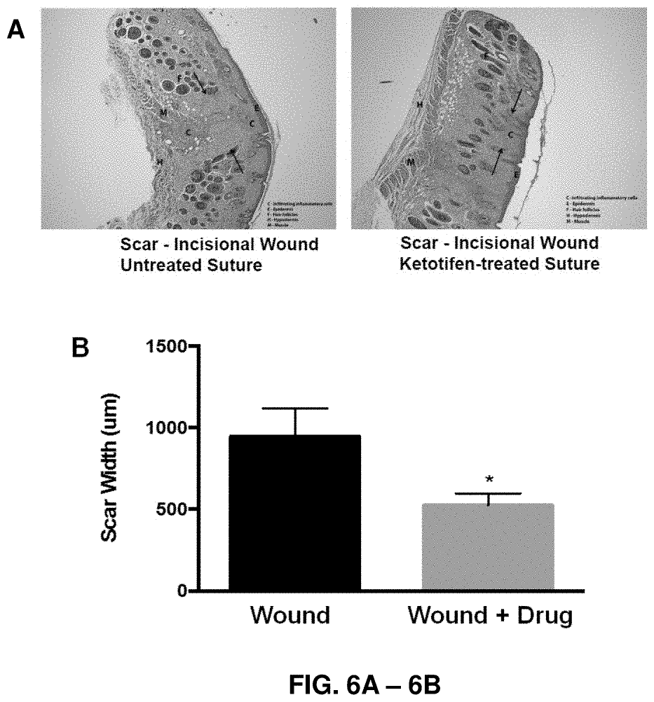

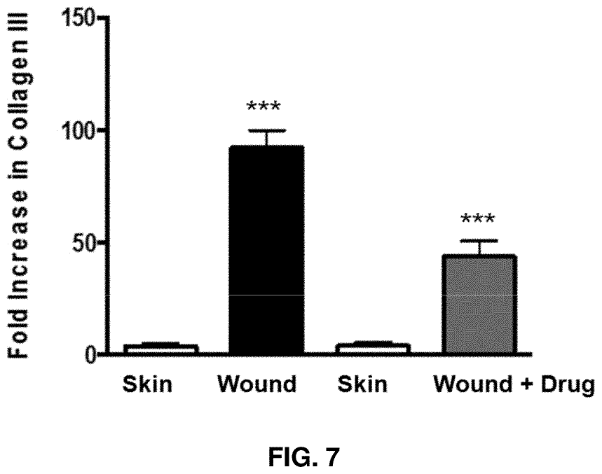

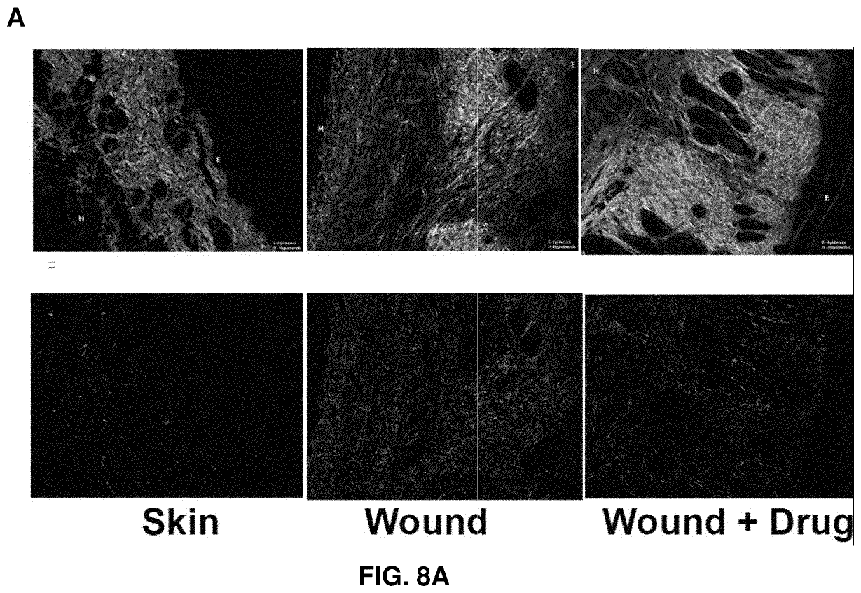

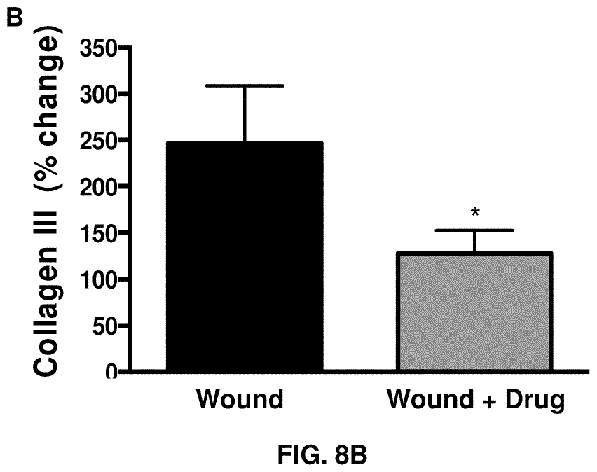

Treatment with ketotifen lessened the inflammatory response to the incisional wound as shown for day 7 (FIG. 5). At day 14, scar width was significantly less in the mice treated with ketotifen (FIG. 6). The relative abundance of newly synthesized collagen III mRNA in excised wound scar (14-day) was also significantly less in the drug group compared to untreated, as determined by q-PCR (FIG. 7). This finding was further confirmed by analyzing excised wound scar with birefringence microscopy. The birefringence of newly synthesized collagen III (green) differs from pre-existing collagen I (red) and is less abundant in wound scar compared to untreated wound scar (FIG. 8A). This analysis showed that less newly synthesized collagen III is found in wound scar from mice treated with ketotifen eluted from suture compared to those wound scars treated with suture alone (FIG. 8B).

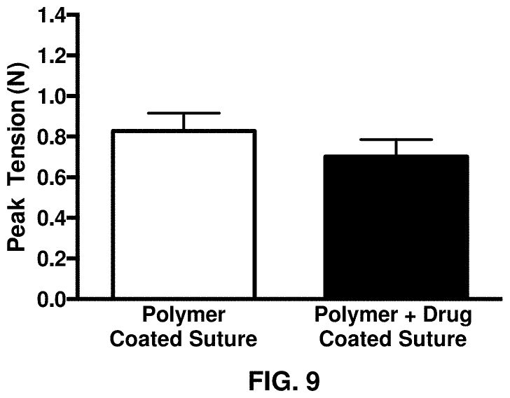

Next, peak tension (measured in Newtons) was determined on excised wound scars from ketotifen-treated and untreated mice. Tensile strength of the scar was not affected by treating with a mast cell stabilizer (FIG. 9).

Collectively these results demonstrate that administering a mast cell stabilizer directly and continually into the wound during the wound healing process decreases the amount of newly synthesized collagen in the scar but does not compromise the strength of the scar. Mast cells are not necessary for incisional wound closure or scar formation, but do contribute to the amount of collagen in the scar. Taken together, it is proposed that the local delivery of therapeutics that prevent mast cell degranulation should minimize the fibrotic scar phenotype. The concept of the disclosed method is basically repurposing ketotifen or other canonical mast cell stabilizers for local and continuous delivery to a wound during the healing process. In the disclosed experiments ketotifen was eluted from wound closures but could just as easily be applied as a cream, gel or eluted from bandage. A topical formulation may also be useful to reduce itch and pain associated with keloids and hypertrophic scars.

### Example 4

### HIF Prolyl Hydroxylase Inhibitor Inhibits Fibroblast Contracture

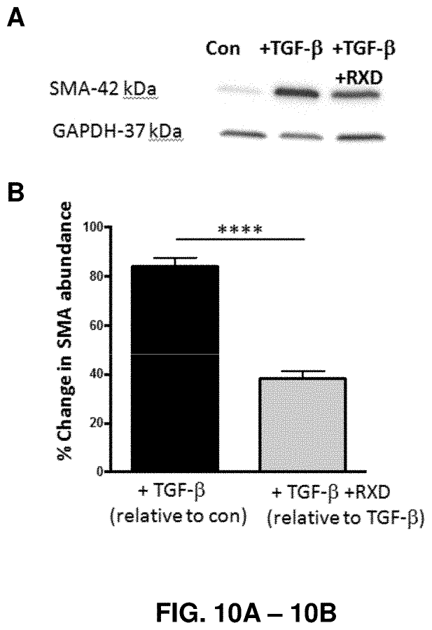

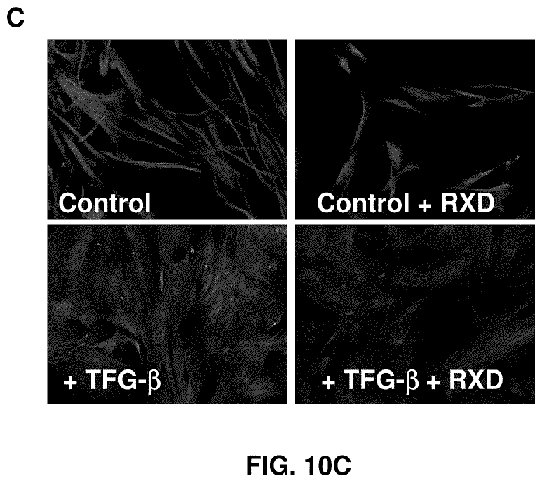

Scar contracture is a painful aspect of hypertrophic scarring due to increased SMA in the fibroblasts. During the inflammatory phase of wound healing, the cytokine transforming growth factor β (TGF-β) is secreted and can activate fibroblasts to produce SMA. Using the HIF prolyl hydroxylase inhibitor roxadustat (RXD) the inventors demonstrate by Western blot (FIG. 10A-10B) that the abundance of TGF-β induced SMA in fibroblasts is inhibited (FIG. 10C).

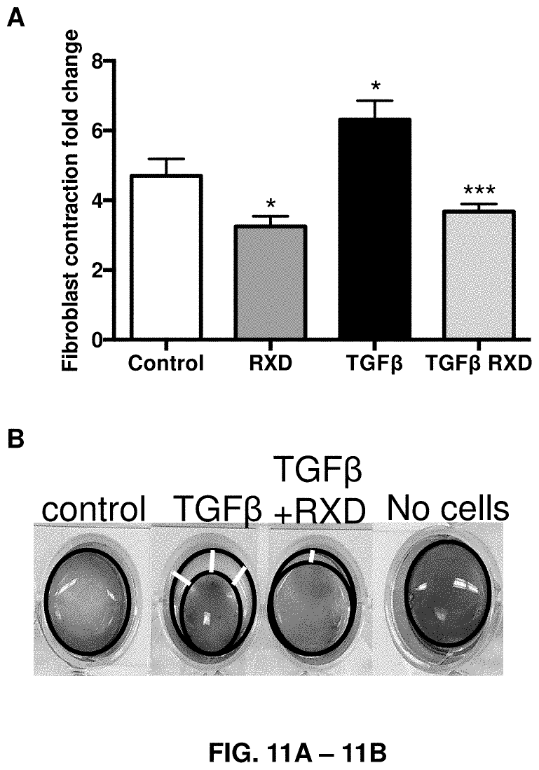

The action of RXD is further seen in its effect on fibroblasts in an in vitro gel contraction assay (FIG. 11). The addition of TGF-β to the fibroblasts seeded on the gel leads to a significant increase in contracture which is inhibited with RXD (FIG. 11A-11B) (TGF-β) as quantified in FIG. 11A. This indicates that RXD is blocking the SMA-induced contraction of fibroblasts.

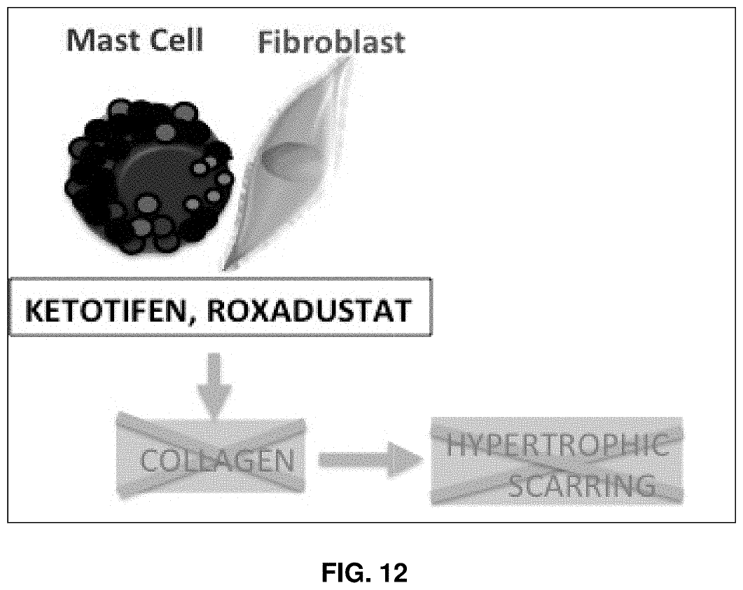

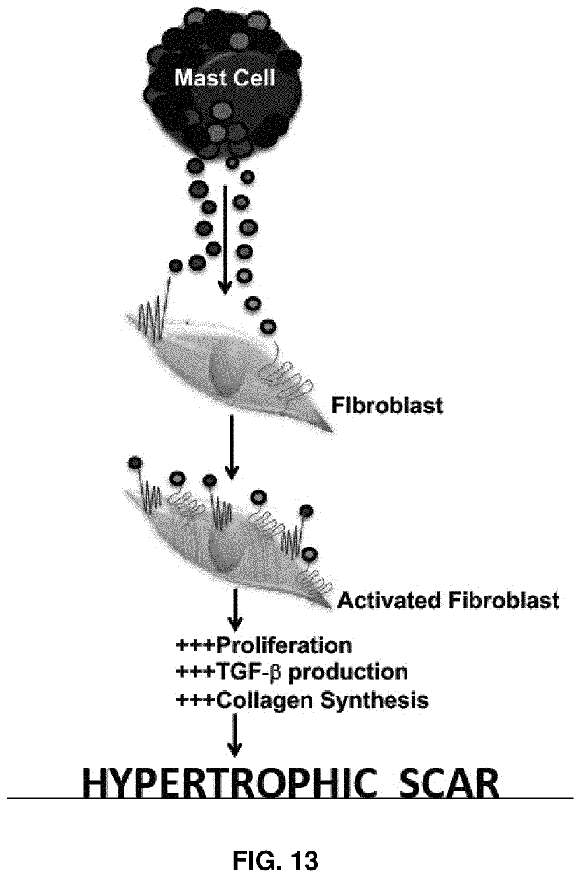

Collectively, this disclosure provides that mast cell stabilizers and HIF prolyl hydroxylase domain inhibitors can be used to to reduce collagen abundance, width, and scar tissue contracture. In addition, this disclosure provides dual therapeutic approach targeting both mast cells and fibroblasts with a mast cell stabilizer such as ketotifen, delivered locally to an incision, along with the HIF prolyl hydroxylase domain inhibitor RXD, as means to reduce collagen abundance, width, and scar tissue contracture. FIG. 12 shows a schematic describing the use of ketotifen and/or roxadustat to inhibit hypertrophic scarring. These therapeutics used alone or in combination will minimize abnormal scarring, itch, pain and contracture (FIG. 13).

What is claimed is:

1. A method for reducing scar collagen abundance, scar width, and scar tissue contracture comprising administering to a subject in need of such treatment an effective amount of a mast cell stabilizer.

2. The method for claim 1, wherein the mast cell stabilizer is selected from the group consisting of cromoglicic acid, ketotifen, olopatadine, rupatadine, mepolizumab, omalizumab, pemirolast, quercetin, nedocromil, azelastine, tranilast, palmitoylethanolamide, and vitamin D.

3. The method for claim 2, wherein the mast cell stabilizer is ketotifen.

4. The method for claim 1, wherein the mast cell stabilizer is administered locally to a wound.

5. A method for reducing scar collagen abundance, scar width, and scar tissue contracture comprising administering to a subject in need of such treatment an effective amount of a HIF prolyl hydroxylase domain inhibitor.

6. The method for claim 5, wherein the HIF prolyl hydroxylase inhibitor is selected from the group consisting of Roxadustat (RXD) (FG-4592), Vadadustat (AKB-6548), Daprodustat (GSK-1278863), and Molidustat (BAY 85-3934).

7. The method for claim 6, wherein the HIF prolyl hydroxylase inhibitor is RXD.

8. The method for claim 7, wherein RXD is administered systemically.

9. A method for reducing scar collagen abundance, scar width, and scar tissue contracture comprising administering to a subject in need of such treatment a combination therapy comprising an effective amount of a mast cell stabilizer and an effective amount of a HIF prolyl hydroxylase domain inhibitor.

10. The method for claim 9, wherein the mast cell stabilizer and the HIF prolyl hydroxylase domain inhibitor are administered as one composition.

11. The method for claim 9, wherein the mast cell stabilizer and the HIF prolyl hydroxylase domain inhibitor are administered consecutively.

12. The method for claim 9, wherein the mast cell stabilizer and the HIF prolyl hydroxylase domain inhibitor are administered separately.

13. The method for claim 9, wherein the mast cell stabilizer and the HIF prolyl hydroxylase domain inhibitor are administered simultaneously.

14. The method for claim 9, wherein the mast cell stabilizer is selected from the group consisting of cromoglicic acid, ketotifen, olopatadine, rupatadine, mepolizumab, omalizumab, pemirolast, quercetin, nedocromil, azelastine, tranilast, palmitoylethanolamide, and vitamin D.

15. The method for claim 14, wherein the mast cell stabilizer is ketotifen.

16. The method for claim 9, wherein the mast cell stabilizer is administered locally to an incision site.

17. The method for claim 9, wherein the HIF prolyl hydroxylase inhibitor is selected from the group consisting of Roxadustat (RXD) (FG-4592), Vadadustat (AKB-6548), Daprodustat (GSK-1278863), and Molidustat (BAY 85-3934).

18. The method for claim 17, wherein the HIF prolyl hydroxylase inhibitor is RXD.

19. The method for claim 9, wherein the HIF prolyl hydroxylase inhibitor is administered systemically.
